(19) United States
(12) Patent Application Publication
(10) Pub. No.: US 2006/0008483 A1
(43) Pub. Date: Jan. 12, 2006

(54) Dispersions of lipids for use as therapeutic and cosmetic agents and intracellular delivery vehicles

(75) Inventors: Frederic Henot, Bruxelles (BE); Roland Frebillot, Six Fours Les Plages (FR); Sebastien Baudart, Tourpes (FR); Thierry Legon, Korbeek-Lo (BE)

Correspondence Address: CLARK & ELBING LLP, 101 FEDERAL STREET, BOSTON, MA, 02110, US

(73) Assignee: Biotech Tools SA, Bruxelles (BE)

(21) Appl. No.: 10/513,736

(22) PCT Filed: May 6, 2003
(86) PCT No.: PCT/BE03/00080

§ 371 Date: Apr. 25, 2005

Related U.S. Application Data
(63) Continuation of application No. 10/140,369, filed on May 7, 2002, now abandoned.

(51) Int. Cl. A61K 8/37; A61K 9/127

(52) U.S. Cl. 424/401; 424/450

(57) ABSTRACT

In a dispersion of lipids in a dispersing medium, the said lipids comprising an amino-amidine compound A, the amidine function of the said compound A is titrated substantially in water by means of an acid HX, wherein X is an anion, in a manner such that the pH of the said lipid dispersion is between about 6.5 and 7.8 within a temperature range from about 2° C. to 40° C. The so titrated dispersion is useful inter alia as a component of a synthetic vector for therapeutic molecules or macromolecules.

The present invention relates to non-viral delivery systems for therapeutic agents. More specifically this invention relates to lipid dispersions which optimal transfection of biomolecules into cells. The present invention further relates to a method for making such dispersions and for making solid compositions therefrom. These dispersions are useful as components of synthetic vectors for therapeutic molecules or macromolecules such as DNA, proteins and polypeptides and therefore useful for introducing such molecules into eukaryotic cells. The invention also relates to cells transformed by means of such synthetic vectors as well as to pharmaceutical compositions comprising effective amounts thereof.

### BACKGROUND OF THE INVENTION

Liposomes may be defined as vesicles in which an aqueous volume is entirely enclosed by a bilayer membrane composed of lipid molecules. When dispersing these lipids in aqueous media, a population of liposomes with sizes ranging from about 15 nm to about 1 μm may be formed. The three major types of lipids, i.e. phospholipids, cholesterol and glycolipids, are amphipathic molecules which, when surrounded on all sides by an aqueous environment, tend to arrange in such a way that the hydrophobic “tail” regions orient toward the center of the vesicle while the hydrophilic “head” regions are exposed to the aqueous phase. According to this mechanism liposomes thus usually form bilayers.

Several types of liposomes are known in the art. Referring to their physical structure, the more simple type of liposomes to prepare consists of multilamellar vesicles (hereinafter referred to as MLV, according to standard practice in the art), i.e. onion-like structures characterized by multiple membrane bilayers, each separated from the next by an aqueous layer, usually having a size between about 100 nm and 1 μm. Their production can be reproducibly scaled-up to large volumes and they are mechanically stable upon storage for long periods of time. Contrary to this, small unilamellar vesicles (hereinafter referred to as SUV, according to standard practice in the art) usually having a size between about 15 nm and 200 nm, possess a single bilayer membrane and are usually difficult to prepare on a large scale because of the high energy input required for their production and of the risks of oxidation and hydrolysis. In addition, SUV are thermodynamically unstable and are susceptible to aggregation and fusion. Furthermore, as the curvature of the membrane increases in SUV, it develops a degree of asymmetry, i.e. the restriction in packing geometry dictates that significantly more than 50% and up to 70% of the lipids making up the bilayer is located on the outside. Because of this asymmetry, the behaviour of SUV is markedly different from that of bilayer membranes comprising MLV or from that of large unilamellar vesicles (the latter, hereinafter referred to as LUV, usually having a size between about 100 nm and 1 μm).

Referring to their chemical structure, liposomes may be made from neutral phospholipids, negatively-charged (acidic) phospholipids, sterols and other non-structural lipophilic compounds. For instance, EP-B-185,680 discloses steroidal liposomes comprising completely closed bilayers substantially comprising a salt form of an organic acid derivative of a sterol. A population of detergent-free liposomes having a substantially unimodal distribution (i.e. unilamellar vesicles) about a mean diameter greater than 50 nm and exhibiting less than a two-fold variation in size may be produced, according to EP-B-185,756, by first preparing multilamellar liposomes and then repeatedly passing the liposomes under pressure through a filter having a pore size not more than 100 nm. Multilamellar vesicles are known from U.S. Pat. No. 4,522,803 and U.S. Pat. No. 4,558,579. A process for improving the trapping efficiency of multilamellar vesicles, comprising repeated freezing at −196° C. and warming in a constant temperature bath, is also disclosed by EP-B-231,261. For a detailed description of liposomes and methods of manufacturing them, reference is hereby made to Liposomes, a practical approach (1990), Oxford University Press. WO 89/03679 discloses the production of liposomes comprising the salt form of a pH sensitive lipid being an organic acid derivative of a sterol or a tocopherol. WO 95/17378 discloses positively charged vesicles for combination with nucleic acids, polypeptides or proteins, comprising a compound having an amidine group. More specifically, this document shows efficiencies of 60 to 68% when transfecting Chinese hamster Ovary cells or K562 human myeloid cells by means of MLV consisting of 3-tetradecylamino-N-terbutyl-N′-tetradecylpropionamidine. However, it appears that certain other cell lines, for instance fibroblasts such as COS-7 monkey fibroblasts or NIH-3T3 mouse fibroblasts, are not appropriately transfected by means of MLV comprising the amino-amidine compounds of the prior art. This indicates that, within the family of amino-amidine compounds used for preparing vesicles for intracellular delivery of genetic material into eukaryotic cells, a need exists in the art for additional modifications of the said compounds, the physical structure of vesicles including them, and/or their dispersions, allowing for appropriate transfection of a wide range of cells, including certain types of cells such as the above-mentioned fibroblasts, and optionally rendering the vesicles sensitive to the pH shift occurring during their introduction into the cell. A need also exists in the art for producing new types of dispersions comprising such compounds which would be mechanically stable upon storage for long periods of time while retaining their pH-sensitivity characteristics.

Attempts have been made to produce vesicles from the amino-amidine compounds of the prior art in the presence of an organic hydrophobic polyfunctional buffer for instance taken from the classes of aminosulfonic acids or hydroxylated amines. For instance, Defrise-Quertain et al. in J. Chem. Soc., Chem. Commun. (1986) 1060-1062 discloses producing dispersions, similar to liposome suspensions and having a bilayer organization, by vortex mixing 3-tetradecylamino-N-terbutyl-N′-tetradecylpropionamidine (having a melting point of 34° C.) in hot water (40-70° C.) optionally in the presence of a tris(hydroxymethyl)aminoethane/HCl buffer and, upon sonication, decreasing the hydrodynamic diameter of the vesicles down to 87 nm. Unfortunately, repeating this manufacturing procedure has evidenced the problems of (i) yielding dispersions of which the pH is not compatible with physiological pH within the temperature range useful for most medical applications, and (ii) yielding dispersions which readily precipitate after a few hours storage at low temperature. Pector et al. in Biochimica et Biophysica Acta (1998) 339-346 discloses forming liposomes of 3-tetradecylamino-N-terbutyl-N′-tetradecylpropionamidine after addition of a buffer comprising N-2-hydroxyethylpiperazine-N′-2-ethanesulfonic acid at pH 7.3 and mechanical mixing above 23° C., then titrating the said liposomes at various saline concentrations by means of 0.1 M HCl. Due to the fact however that the organic buffer anion being present in the latter procedure is able to interact with any positively charged amidino group, the disclosed procedure necessarily achieves a poorly defined mixture of lipidic particles that is not suitable for further handling, storage and use as intracellular delivery vehicles, therefore teaching away from the manufacture of vectors introducing molecules and macromolecules into a cell. Therefore there is a need in the art for well-defined dispersions, e.g. cationic liposomes or micelles, based on compounds having an amidine function that would be suitable as intracellular delivery vehicles. There is furthermore a need in the art for lipid dispersions that combine stability during production with optimal transfection efficiency. In particular, there is a need in the art for such aqueous dispersions which would have a pH compatible with physiological pH within the temperature range useful for most medical applications, e.g. between about 2° C. and 40° C. There is also a need in the art for dispersions which combine such an amino-amidine compound with another type of lipid while keeping the above-mentioned advantageous characteristics. For economical reasons, such as decreasing the volume and weight of the useful biological material and hence its cost of transportation, there is also a need in the art for solid compositions such as lyophilisates or amorphous particles, which are able to achieve and retain the above-mentioned advantageous characteristics when dispersed in a liquid medium such as water or an appropriate buffer.

### SUMMARY OF THE INVENTION

The present invention is based on several unexpected findings. First, compounds having an amino-amidinium function, when titrated with an acid under particular conditions, result in well-defined lipid dispersions, which are capable of retaining a pH compatible with physiological pH within a temperature range useful for most medical applications, e.g. between about 2° C. and 40° C. Another aspect of the invention is the ability of the said dispersions based on compounds having an amidine function to efficiently transfect in vitro or in vivo a wide range of types of cells. Additionally and quite importantly, the pH characteristics of the lipid dispersions of the invention are not adversely affected when the said dispersions are dried or freeze-dried into a solid composition and the said solid composition is thereafter re-dispersed in another aqueous medium such as for instance an organic functional buffer.

A further aspect of the invention is the observation that compounds of the present invention under certain circumstances have an unexpectedly high CMC (in the order of 10⁻³ to 10⁻⁶M) which make it possible to work with dispersions at a concentration around the critical micellar concentration (CMC), combining stability of the dispersion with optimal transfection efficiency. Thus, according to a preferred embodiment of the present invention, the composition of the lipid dispersion of amino-amidines defined as compound A will be such that the concentration is close to the CMC value.

According to a third aspect of the invention, it was found that association of amino-amidinium group with a cosmotropic anion, most preferably a phosphate anion, will improve the stability of the lipid dispersions which is beneficial for its various applications.

The improved dispersions of the present invention have a number of applications in the medical, cosmetic and industrial fields.

Moreover, the dispersions of the present invention are useful for making synthetic vectors for combining with a wide range of biologically active molecules, especially for complexing or entrapping macromolecular and/or biodegradable substrates. Additionally it was found that the characteristics of such complexes can be further improved by the addition of block polymer surfactants.

Further, the resulting synthetic vectors are effective for introducing the said biologically active molecule or macromolecule into a wide range of eukaryotic cells. This invention further includes methods for introducing biologically active molecules into eukaryotic cells, as well as eukaryotic cells transformed by means of the aforesaid synthetic vectors and pharmaceutical compositions comprising effective amounts of the synthetic vectors. The said pharmaceutical compositions are useful for the prophylactic or therapeutic treatment of mammals for a wide range of diseases and disorders, depending on the biological activity of the relevant molecule or macromolecule.

According to a first aspect of the invention, a dispersion of particles is provided comprising compound A having the general formula:
R₁HN—(CH₂)ₙ—C(═NR₂)—NR₃R₄ (I)
wherein each of R₁, R₂, R₃ and R₄ is independently selected from the group consisting of hydrogen, C₃₋₂₀ alkyl, C₃₋₁₀ cycloalkyl, aryl and heteroaryl radicals, preferably R₁ and R₃ are a C₃₋₂₀ alkyl, most preferably R₁ is a C12, C14, C16 or C16 alkyl, and R₂ is an isopropyl or a terbutyl and R₄ is a hydrogen;
wherein n is an integer from 1 to 6 inclusive, and
wherein the amidine function of the said compound A is titrated substantially in water by means of an acid HX, wherein X is an anion, in a manner such that the pH of the said lipid dispersion is between about 6.5 and 7.8 within a temperature range from about 2° C. to 40° C.

According to a preferred embodiment said compound A is a compound having the general formula (I) wherein and R₂ is an isopropyl or terbutyl.

According to a further embodiment of the invention the dispersion of compound A further comprises another lipid B. Thus the present invention further relates to lipid dispersions, comprising compound A and one or more other lipids.

According to a further embodiment of the invention anion X is a cosmotropic anion, preferably selected from the group comprising phosphate, sulphate, citrate, hydrogen-phosphate, and most preferably a phosphate. The invention thus also relates to a salt obtained by titration of the compound of formula (I) with a cosmotropic anion and to dispersions thereof, optionally additionally comprising another lipid B.

The present invention further describes a method of making the lipid dispersions described above, the method comprising
(a) dispersing an amino-amidine compound A having the general formula as described above optionally admixed with one or more lipids B, in a liquid medium comprising an aqueous medium and optionally an organic solvent for compound A, and optionally an oily component,
(b) optionally processing the dispersion obtained in step (a), until vesicles comprising compound A and optionally one or more lipid(s) B are obtained, and
(c) titrating the vesicles obtained in step (a) or (b) with an acid HX, wherein X is an anion, and
(d) optionally processing the titrated dispersion obtained in step (c) until pH stabilisation
whereby the method is characterized in that:
   the aqueous medium of step (a) substantially consists of water, and
   the acid HX is used in step (c) in an amount such as to substantially form an amidinium salt (A, HX) and such that the pH of the said lipid dispersion after titration is between about 6.5 and 7.8, preferably between 7.0 and 7.6 within a temperature range from about 2° C. to 40° C.

According to another aspect of the invention, the lipid dispersion of compound A defined by formula (I), as detailed above, optionally comprising another lipid B, is a dispersion comprising micelles of the amphiphatic compounds of the invention, i.e. the concentration of compound A or the amidinium salt in the dispersion is close to or equal to the CMC, or provided in a concentration which upon dilution or concentration for application is around the CMC. Moreover, according to a particular aspect of the invention, compounds are added to the dispersion which improve the stability thereof. According to a particular embodiment of the invention, such compounds are polymeric (poly)cations, which are added directly to the lipid dispersion.

According to this aspect of the invention, the method for producing the lipid dispersion of the invention may either be performed under particular circumstances and at a concentration of compound A around the CMC or may include an additional step comprising the concentration or dilution of the dispersion in order to obtain a micellar dispersion.

According to a further aspect of the invention the improved dispersion of the present invention can be of use for instance in its applications as an anti-microbial agent, an anti-inflammatory agent, a cosmetic agent, an emulsifier, a detergent, a vaccine adjuvant or a diagnostic reagent.

According to another aspect of the invention the described dispersion of particles is used for the production of vectors by combination with a wide range of biologically active molecules, especially for complexing or entrapping macromolecular and/or biodegradable substrates. The biologically active molecules of the present invention include, but are not limited to (single or double stranded) DNA, RNA, DNA-RNA hybrids, proteins and peptides, polynucleotide-protein/peptide hybrids and small molecules. A further aspect of the invention is the addition of polymeric anions further stabilizing the complex between lipids and DNA.

Accordingly, the present invention further relates to vectors comprising a complex between an amino-amidine compound A as described above and at least one biologically active molecule, preferably a polynucleotide.

### BRIEF DESCRIPTION OF THE DRAWINGS

FIG. 1 shows the variation of pH at 25° C. as a function of the amount of hydrochloric acid added during titration of an amino-amidine dispersion in water (injection grade available from Baxter, cat. Nr. ADA 0304).

FIG. 2 shows the variation of pH at 25° C. as a function of the amount of hydrochloric acid added during titration of an amino-amidine dispersion in sodium phosphate buffer.

### DETAILED DESCRIPTION OF THE INVENTION

“Transfection” is defined herein as the intracellular delivery of active biological material, especially genetic material (such as defined hereinafter) into the cells of a eukaryotic organism, preferably a mammal, and more preferably, a human. The said genetic material is preferably expressible (with or without integration into the genome) and produces beneficial proteins after being introduced into the cell. Alternatively, the said genetic material is used to bind to or interact with a site within the cell, or encodes a material that binds to or interacts with a site within the cell. Suitable cell types that can be transfected using this invention include, but are not limited to, cells performing endocytosis or phagocytosis, fibroblasts, myoblasts, hepatocytes, cells of hematopoetic origin such as white blood cells and bone marrow cells, cancer cells and ischemic tissue. Transfection can be performed in vitro, ex vivo, or in vivo. The genetic material can be transiently expressed or stably expressed.

In vitro transfection involves transfecting cells outside of a living eukaryotic organism, e.g. using cell cultures. In vivo transfection involves transfecting cells within a living eukaryotic organism. Ex vivo transfection involves removing cells from an organism, transfecting at least part of the cells, and returning the cells to the said organism.

The term “removing” as used herein refers to any method known to obtain a sample of living cells from a eukaryotic organism, including venipucture, cell scraping, punch biopsy, needle biopsy and surgical excision. The term “returning” includes methods known to replace cells in the body of a mammal, preferably a human, such as intravenous introduction, surgical implantation and injection.

“Transient gene expression” is defined herein as temporary gene expression that diminishes over time under selective conditions, i.e. usually occurring over periods of less than one year to periods as short as one week. The gene therapy application, the vector construct, whether or not chromosome integration has occurred, the cell type and the location of cell implantation following transfection are known to influence the length of time that a particular gene is expressed.

“Stable gene expression” is defined herein as gene expression that does not significantly diminish over time, i.e. the transfected cells produce a relatively constant level of gene product for relatively long periods of time.

“Genetic material” is defined herein as DNA, RNA, mRNA, rRNA, tRNA, uRNA, ribozymes, antisense oligonucleotides, peptide nucleic acid (PNA), plasmid DNA or a combination thereof. Modified nucleosides can be incorporated into the genetic material in order to impart in vivo and in vitro stability of the oligonucleotides to endo- and exonucleases, alter the charge, hydrophilicity or lipophilicity of the molecule, and/or provide differences in three-dimensional structure.

As used herein, the term “C₃₋₈ alkyl” refers to straight and branched chain saturated hydrocarbon monovalent radicals or groups having from 3 to 8 carbon atoms such as, for example, propyl, n-butyl, 1-methylethyl, 2-methylpropyl, 1,1-dimethylethyl, 2-methylbutyl, n-pentyl, dimethylpropyl, n-hexyl, 2-methylpentyl, 3-methylpentyl, n-heptyl, 2-ethylhexyl, n-octyl and the like.

As used herein, the term “C₃₋₁₀ cycloalkyl” refers to monocyclic or polycyclic aliphatic monovalent radicals or groups having from 3 to 8 carbon atoms, such as for instance cyclopropyl, 1-2-dimethylcyclopropyl, cyclobutyl, methylcyclobutyl, cyclopentyl, methylcyclopentyl, cyclohexyl, methylcyclohexyl, ethylcyclohexyl, cycloheptyl, cyclooctyl, norbornyl, adamantyl, and the like.

As used herein, the term “aryl” refers to mono- and polyaromatic monovalent radicals such as phenyl, naphtyl, anthracenyl, phenantracyl, fluoranthenyl, chrysenyl, pyrenyl, picenyl and the like, including fused benzo-C₅₋₈ cycloalkyl radicals such as, for instance, indanyl, 1,2,3,4-tetrahydronaphtalenyl, fluorenyl and the like.

As used herein, the term “heteroaryl” means a mono- and polyheteroaromatic monovalent radical including one or more heteroatoms selected from the group consisting of nitrogen, oxygen, sulfur and phosphorus, such as for instance pyridinyl, pyrazinyl, pyrimidinyl, pyridazinyl, triazinyl, triazolyl, imidazolyl, pyrazolyl, thiazolyl, isothiazolyl, oxazolyl, pyrrolyl, furanyl, thienyl, indolyl, indazolyl, benzofuryl, benzothienyl, quinolyl, quinazolinyl, quinoxalinyl, carbazolyl, phenoxazinyl, phenothiazinyl, xanthenyl, purinyl and the like, including all possible isomeric forms thereof.

As used herein, the term “C₁₂₋₂₀ alkyl” refers to straight and branched chain saturated or unsaturated hydrocarbon monovalent radicals having from 12 to 20 carbon atoms such as, for example, dodecyl, tetradecyl, hexadecyl, octadecyl and the like.

As used herein, the term “C₃₋₂₀ alkyl” includes C₃₋₈ alkyl and C₁₂₋₂₀ alkyl (such as hereinabove defined) and homologues thereof having from 9 to 11 carbon atoms, such as for instance nonyl, decyl, undecyl and the like.

In a first embodiment, the present invention includes a lipid dispersion comprising an amino-amidine compound A having the general formula:
R₁HN—(CH₂)ₙ—C(═NR₂)—NR₃R₄ (I)
wherein each of R₁, R₂, R₃ and R₄ is independently selected from the group consisting of hydrogen, C₃₋₂₀ alkyl, C₃₋₁₀ cycloalkyl, aryl and heteroaryl radicals, and n is a positive integer, the said dispersion being characterized in that the amidine function of the said compound A is titrated substantially in water by means of an acid HX, wherein X is an anion, in a manner such that the pH of the said lipid dispersion is between about 6.5 and 7.8 within a temperature range from about 2° C. to 40° C.

In view of the main uses of the dispersion of the invention, such as detailed hereinafter, it is highly preferred that:
R₂ is a C₃₋₈ alkyl group, preferably an isopropyl or terbutyl group,
n is an integer from 1 to 6 inclusive, preferably n=2, and
R₁ is a C₁₂₋₂₀ alkyl group, one of R₃ and R₄ is hydrogen and R₃ and R₄ is a C₁₂₋₂₀ alkyl group.

In a most preferred embodiment of this invention, the amino-amidine compound A is selected from the group comprising N-terbutyl-N′-tetradecyl-3-tetradecyl-aminopropionamidine, N-terbutyl-N′-dodecyl-3-dodecylamino-propionamidine, N-terbutyl-N′-hexadecyl-3-hexadecylaminopropionamidine and N-terbutyl-N′-octadecyl-3-octadecylaminopropionamidine, N-isopropyl-N′-tetradecyl-3-tetradecyl-aminopropionamidine, N-isopropyl-N′-dodecyl-3-dodecylamino-propionamidine, N-isopropyl-N′-hexadecyl-3-hexadecylaminopropionamidine and N-isopropyl-N′-octadecyl-3-octadecylaminopropionamidine. All amino-amidine compounds A falling under the above definition, especially those mentioned in the above most preferred embodiment, are either well known in the art or can be obtained by procedures and methods similar to the procedures used for preparing the well known compounds (i.e. by aminolysis of ethyl-N-terbutyl- of etyl-N-isopropyl-acrylimidate with a fatty amine, see for instance D. G. Neilson ‘The Chemistry of Amidines and Imidates’ (1975), ed. S. Patai, Wiley, New-York, and R. Fuks, Bull. Soc. Chim. Belg. (1980) 89:433), while performing routine experimental work and changing the starting materials according to ordinary skill in the art.

The dispersion according to the first embodiment of the invention may include only, i.e. may consist of, an amino-amidine compound A having the general formula (I) or a mixture of such compounds, or alternatively it may further include one or more lipid(s) B. Such lipids may for instance be selected from the group consisting of phospholipids, sterols, tocopherols or other lipophilic compounds, preferably those which are already known in the art for their ability to form liposomes or micelles under appropriate conditions. Preferably the lipid B is a biocompatible lipid selected from the group consisting of fatty acids, lysolipids, phosphatidylcholines, phosphatidylethanolamines, phosphatidylserines, phosphatidylglycerols, phosphatidylinositols, sphingolipids (such as sphingomyelin), glycolipids (such as gangliosides), sulfatides, glycosphingolipids; lipids bearing functional moieties such as polyethyleneglycol, chitin, hyaluronic acid, polyvinylpyrrolidone, polylysine, polyarginine, sulfonated mono-, di- or oligosaccharides; cholesterols; sterol aliphatic acid esters (such as cholesterol butyrate, cholesterol isobutyrate, cholesterol palmitate, cholesterol stearate, lanosterol acetate, ergosterol palmitate, and phytosterol n-butyrate); dicetyl phosphates, stearylamines, cardiolipin, synthetic phospholipids with asymmetric acyl chains, ceramides, sterol aliphatic acid esters; sterol esters of sugar acids (such as cholesterol glucuronide, lanosterol glucuronide, 7-dehydrocholesterol glucuronide, ergosterol glucuronide, cholesterol gluconate, lanosterol gluconate, and ergosterol gluconate); esters of sugar acids and sugar alcohols (such as lauryl glucuronide, stearoyl glucuronide, myristoyl glucuronide, lauryl gluconate, myristoyl gluconate, and stearoyl gluconate); sugar esters and aliphatic acid esters (such as sucrose laurate, fructose laurate, sucrose palmitate, sucrose stearate, glucuronic acid, gluconic acid, accharic acid, and polyuronic acid); saponins (such as sarsasapogenin, smilagenin, hederagenin, oleanolic acid, and digitoxigenin); glycerol esters (such as glycerol tripalmitate, glycerol distearate, glycerol tristearate, glycerol dimyristate, and glycerol trimyristate); alcohols having 10 to 30 carbon atoms (such as n-decyl alcohol, lauryl alcohol, myristyl alcohol, cetyl alcohol, and n-octadecyl alcohol), D-galactopyranosides, digalactosyldiglyceride, N-succinyldioleoylphosphatidyl-ethanolamine, palmitoyl homocysteine, alkyl phosphonates, alkyl phosphinates and alkyl phosphites. Suitable phosphatidylcholines include dioleoylphosphatidylcholine, dimyristoylphosphatidylcholine, dipentadecanoyl-phosphatidylcholine, dilauroylphosphatidylcholine, dipalmitoylphosphatidyl-choline and distearoylphosphatidylcholine.

Thus, according to a particular embodiment, the lipid dispersion of the present invention can further comprise one or more lipids ‘B’. The lipid(s) B may be admixed with the amino-amidine compounds A in various proportions. The only restriction to the selection of the lipids B and of their proportion in the mixture is that they should not significantly affect or otherwise be detrimental to the advantageous properties of the lipid dispersions of the invention, i.e. retaining their pH characteristics (such as above defined) while being able to efficiently transfect a wide range of types of cells. Given the teachings of the present invention and the general knowledge in the art, the skilled person will be able in each case to determine whether a given lipid B and a given proportion for the latter meet these criteria. However, as a general rule, it should be understood that the amino-amidine compound A should preferably be the main organic component of the dispersion of the invention, i.e. the molar amount of the lipid(s) B should preferably be at most about 50%.

Titration of the amidine function of compound A substantially in water is an essential requirement of this first embodiment of the present invention. The term “substantially in water” as used herein means that, contrary to the teachings of the prior art, titration by means of an acid HX is not effected in the presence of organic functional buffers, such as aminosulfonic acid or hydroxylated amines, which were found to greatly interfere with liposomes stability. Although substantially pure water is a preferred embodiment of the present invention, a mineral buffer such as sodium or potassium phosphate or alkali metal salts of bicarbonate often does not significantly interfere with stability of the lipid dispersion obtained and may therefore be used in place of pure water. Another requirement of the first embodiment of the present invention is that titration of compound A should be performed until the pH of the lipid dispersion is between about 6.5 and 7.8 within a temperature range from about 2° C. to 40° C. Such further condition clearly contributes to obtaining a well chemically defined composition, as opposed to the poorly defined mixtures of salts and liposomes of the prior art. The skilled person knows how to meet this second condition, e.g. by accurately controlling the titration process, for instance by continuously measuring the pH of the dispersion during the addition of the acid HX, by continuously processing the lipid dispersions and by interrupting the said addition as soon as the pH value within the required range is achieved and stable.

The anion X of the acid used for titration of the amidine function according to the first embodiment of the present invention may suitably be either that of a strong acid or a weak acid, these terms being understood according to their usual meaning in the chemical art as exemplified herein-below in a non exhaustive manner. A strong acid includes anions such as iodide, bromide, chloride, nitrate, perchlorate, sulfate, tosylate and methanesulfonate. A weak acid includes anions such as acetate, fluoride, borate, hypobromite, hypochlorite, nitrite, hyponitrite, sulfite, phosphate, phosphite, phosphonate, chlorate, oxalate, malonate, succinate, lactate, carbonate, bicarbonate, benzoate, citrate, permanganate, manganate, propanoate, butanoate and chromate.

According to a preferred embodiment of the present invention however, a cosmotropic anion is used for titration of the amidine function of compound A. A ‘cosmotropic anion’ as used herein refers to an anion which influences the aggregation of proteins in water, as determined by Hoffmeister 1988. Preferably, according to the present invention, a cosmotropic anion is selected from the group comprising of sulfate, phosphate, citrate, and hydrogen-phosphate. Most preferably said cosmotropic anion is a phosphate.

The present invention relates to lipid dispersions of compound A as defined herein, wherein the lipids may take various forms. According to one aspect of the invention, the lipids within the said dispersion are essentially present in the form of micelles. A micelle is formed spontaneously when amphiphilic compounds are present in water above a certain concentration, referred to as the critical micellar concentration (CMC). This is sometimes expressed as Kn=cn/c1n, as the equilibrium constant for the association of monomers. The CMC of compound A of the present invention is a function of the temperature and of the ionic strength of the dispersion medium (buffer) and can be influenced by the presence of other compounds. Thus, according to this aspect of the invention, buffers are used in which the CMC of compound A is preferably between 10⁻³ and 10⁻⁶M, most preferably buffers such as phosphate buffers. According to the present invention obtaining a lipid dispersion at a concentration which is close to the CMC of compound A will improve the stability and transfection efficiency of said lipid dispersion. According to the present invention, additional compounds may be added which improve the stability of the lipid dispersion. Such compounds may be cationic peptides or polymers as further detailed herein. Moreover, compounds that capable of maintaing the osmolarity of the lipid dispersion, such as sugars, can also be added to the lipid dispersion of the invention.

The lipids in the dispersion of the present invention may also be present in the form of liposomes or amorphous solid particles or emulsion droplets. Moreover, the lipid dispersion may be a mixture of different vesicle types and sizes.

Preferably the dispersing medium of the lipid dispersion of the invention is an aqueous medium consisting of water being already present during titration of the amidine function of compound A. As previously mentioned, the dispersing medium may also comprise a mineral buffer which may either be already present during titration of the amidine function or which may be added thereafter if need be for some specific applications. This dispersing medium may also comprise an organic functional buffer. Suitable examples of such organic functional buffers are well known in the art of biology and include for instance aminosulfonic acids (such as N-2-hydroxyethylpiperazine-N′-2-ethanesulfonic acid (HEPES), 3-(N-morpholino)propanesulfonic acid (MOPS), piperazine-N,N′-bis(2-ethanesulfonic acid (PIPES), N-[tris(hydroxymethyl)methyl]-3-amino-2-hydroxypropanesulfonic acid (TAPSO), piperazine-1,4-bis(2-hydroxy propanesulfonic acid) dihydrate (POPSO), N,N-bis(2-hydroxyethyl)-2-aminoethanesulfonic acid (BES), 2-[(2-hydroxy-1,1-bis[hydroxymethyl]ethyl)amino]ethanesulfonic acid (TES), N,N-bis(2-hydroxyethyl)-3-amino-2-hydroxypropanesulfonic acid (DIPSO), [(2-Hydroxy-1,1-bis[hydroxymethyl]ethyl)amino]-1-propanesulfonic acid (TAPS), 3-(cyclohexylamino)-1-propanesulfonic acid (CAPS), 4-(2-hydroxyethyl)piperazine-1-propanesulfonic acid (EPPS), 4-(2-hydroxyethyl)piperazine-1-(2-hydroxypropanesulfonic acid) monohydrate (HEPPSO), 2-(N-morpholino)ethanesulfonic acid and the like), hydroxylated amines (such as tris(hydroxymethyl)aminoethane, N,N-bis(2-hydroxyethyl)glycine (Bicine), N-(2-hydroxy-1,1-bis[hydroxymethyl]ethyl) glycine (Tricine), 1,3-bis[tris(hydroxymethyl) methylamino]propane and the like) and mixtures thereof. According to a particular embodiment of the invention, the buffer is a buffer with a low thermal coefficient, so as not to influence stability of the lipid dispersion upon change of temperature. Buffers with a low thermal coefficient are buffers such as MOPS and phosphate buffers.

The lipids in the dispersions according to the invention may also be present in the form of an emulsion, i.e. for instance by combining a lipid aqueous dispersion with an oily component, the titrated compound A may act as a surfactant and contribute to the formation of a continuous lipid phase surrounded by an amino-amidine layer.

As mentioned above, a number of compounds may be added to the lipid dispersion of the invention to improve the stability and/or the transfection efficiency thereof. For instance, the lipid dispersions of the invention further comprise a (poly)cationic peptide or polymer. Such a polymer is preferably selected from the group comprising total histones, specific histone fractions such as H1, H2, H3, H4, polyglutamine, poly-lysine, mellitin, polymyxin B, spermidine, spermine, DCP68 of chloroplast nucleoids, histone-like proteins including Heat Unstable protein (HU), the Integration Host Factor (IHF) and Histone-like Nucleoid Structuring Protein (H-NS), the inorganic cation Co(NH₃)₆³⁺, and synthetic compounds such as the nitrogen-containing silicones described in U.S. Pat. No. 6,068,980. Most preferably the polycationic peptide is protamine sulfate. According to a preferred embodiment of the present invention the polycation is present in a lipid to polymer weight ratio between 0.1 and 10, preferably between 0.25 and 4, most preferably between 0.5 and 2.

Other compounds that are added to the lipid dispersion of the present invention are compounds which maintain the osmolarity of the lipid dispersion, such as mono-, oligo- or polysaccharides. According to a preferred embodiment said osmolarity is around 260-300 mOsm. Most preferably the added sugar is glucose.

In a further embodiment, the present invention includes a method for making a lipid dispersion such as defined hereinabove, comprising the steps of:
(a) dispersing an amino-amidine compound A having the general formula (I), optionally admixed with one or more lipid(s) B, in a liquid medium comprising an aqueous medium and optionally an organic solvent for compound A and/or B, and optionally an oily component,
(b) optionally processing the dispersion of step (a) until vesicles comprising compound A and optionally one or more lipid(s) B is obtained, and
(c) titrating the dispersion obtained in step (a) or (b) with an acid HX, wherein X is an anion, and
(d) optionally processing the titrated dispersion obtained in step (c) until pH stabilisation
the said method being characterized in that:
   the aqueous medium of step (a) substantially consists of water, and
   the acid HX is used in step (c) in an amount such as to substantially form an amidinium salt (A, HX) and such that the pH of the said lipid dispersion after titration is between about 6.5 and 7.8 within a temperature range from about 2° C. to 40° C.

A first important aspect of the method of the invention is that, contrary to the teachings of the prior art, buffers which due to their chemical definition are likely to interfere with the desired chemical reaction involved during the titration step of the method should be avoided. Interference with titration should be understood to mean that the buffer induces particle aggregation or fusion. In practice, this means that the standard organic multifunctional buffers commonly used in the art of biology, such as the well-known classes of aminosulfonic acids or hydroxylated amines previously described, should be carefully avoided. Therefore the preferred aqueous medium to be used in step (b) of the method is water or, alternatively, a non-interfering buffer as previously disclosed.

A second important feature of the method of the invention is that the acid HX used for titration should be present in an amount sufficient but necessary to substantially and quantitatively form the desired amidinium salt (A, HX), i.e. substantially free of the free-amidine form of compound A. According to a preferred embodiment of the invention the anion X is a cosmotropic anion as defined herein, so as to obtain a salt, upon titration of the amidine function.

An improved working embodiment of the manufacturing method of the invention further includes, after step (c), the step of measuring and optionally adjusting the pH of the titrated dispersion until a pH between about 6.5 and 7.8 is obtained. This optional step may be used as a quality control step and is performed according to standard practice in the art.

Depending on the specific desired form of the lipid dispersion, variations of the manufacturing method of the invention may be as follows. Liposomes or micelles will be obtained when the liquid medium used in step (a) is an aqueous medium, e.g. water or a non-interfering buffer.

If desired, the method of the invention may further include the step of admixing the lipid dispersion obtained after step (c) or step (d) with a buffer. The said buffer may be identical with or different from the mineral buffer (non-adversely interfering with liposome or micellar stability, see above) optionally present during titration. For instance it may be an organic functional buffer such as previously defined.

Another embodiment of the manufacturing method of the invention further includes, after step (c) or (d), and optionally after admixing the lipid dispersion with a buffer (in the latter case, as explained above, a further pH control step is unnecessary in view of the advantageous pH characteristics of the dispersions of the invention), a step (i) of again processing the said lipid dispersion until a predetermined type of vesicles or average size is obtained or until a predetermined size distribution is obtained. When working with liposomes, the processing method of this optional step, alike the processing methods of steps (b) and (d), is well known to those skilled in the art of liposomes and may be selected from any technology disclosed in Liposomes (cited supra), depending on the specific requirements of the further use of the liposomal dispersions, i.e. in particular depending from the desired mean size and mean size distribution of the vesicles or particles in the dispersions. Such processing methods include micro-fluidization, vortex mixing, sonication and the like and make use of conventional manufacturing equipment available in the art. Depending upon the post-processing method selected for step (i), it is possible to achieve either multilamellar vesicles or small unilamellar vesicles or large unilamellar vesicles according to the classification of liposomes provided in the above section “Background of the invention”.

As mentioned above, according to a particular aspect of the invention, the vesicles of the dispersions are micelles. Micellar dispersions are obtained by ensuring that the concentration of the lipids of the invention is around or above the critical micellar concentration (CMC), the CMC itself being dependent on temperature and ionic strength of the medium. Thus, according to a particular embodiment, dispersion of compound A in step (a) of the method of the present invention is performed at A) conditions ensuring a CMC around 10⁻³ to 10⁻⁶M at 25° C. and B) a concentration around the CMC. According to the present invention a concentration around the CMC refers to a concentration of said lipid dispersion which is n×CMC wherein n=0.5-100, preferably n=0.5-10, more preferably n=0.5-5, most preferably n=0.5-2. Thus, a preferred embodiment of the present invention, is a lipid dispersion of N-terbutyl-N′-tetradecyl-3-tetradecyl-aminopropionamidine (DiC14iPr) in PB at a concentration around 5×10⁻⁵M or at 2×10⁻⁵M in PBS+protaminesulfate.

Alternatively, an additional step (i) is provided in the method of the invention as referred to above, which, when working with micelles, comprises concentrating or diluting said dispersion so as to obtain a concentration which is around the CMC. Upon performing this concentration or dilution step care should be taken that the CMC, which itself can be affected by the concentration of the medium is not significantly affected.

A decrease of the CMC can furthermore be achieved by addition of certain compounds to the lipid dispersion. Preferred examples of such compounds are (poly)cationic peptides or polymers compounds as described above. Thus, according to a preferred embodiment the method of obtaining improved lipid dispersions includes the addition of such compounds to the lipid dispersion of the invention to improve the advantageous features thereof.

According to another embodiment, the manufacturing method of the lipid dispersion of the present invention comprises additional steps so as to obtain the dispersion in the form of an emulsion:
(e) drying the titrated and optionally processed vesicles obtained in step (c) or (d) in order to obtain lipid solid particles,
(f) mixing the lipid solid particles obtained in step (e) with an oily component,
(g) raising the temperature of the mixture obtained in step (f) above the melting temperature of the lipid solid particles obtained in step (e) but not until the temperature of degradation of the said lipids (i.e. usually not above about 80° C.), and
(h) emulsifying the mixture obtained in step (g) in the presence of water or a buffer.

Emulsions prepared according to the invention may be of any type, such as oil-in-water emulsions or water-in-oil emulsions.

In order to obtain the lipid dispersion in the form of amorphous solid particles (the latter having the advantages of a well-controlled form and size), another preferred embodiment of the process according to the invention includes the following features:
(j) drying the titrated and optionally processed vesicles obtained in step (c) or (d) or (d′) in order to obtain lipid solid particles,
(k) re-dispersing the solid particles obtained in step (j), optionally admixed with a biologically active molecule and/or with one or more lipids, in an organic solvent for compound A, the said solvent being sparingly miscible with water,
(l) processing the organic dispersion obtained in step (k), and
(m) stripping the organic solvent until amorphous solid particles are obtained.

The skilled person is readily able to select an organic solvent for compound A suitable for carrying out the above embodiment of the process according to the invention. Examples of suitable organic solvents for this purpose include for instance halogenated hydrocarbons such as chloroform and methylene chloride, esters such as ethyl acetate and mixtures thereof. The present invention thus also includes a composition of solid amorphous particles obtainable from the lipid dispersion by this embodiment of the manufacturing method of the invention.

According to yet another embodiment of the manufacturing method, a dried solid composition may be obtained from the dispersion of lipids of the invention by further including a step (n) of drying the titrated and optionally processed vesicles, micelles or liposomes obtained in step (c), (d) or (i). When the drying step (h) is freeze-drying, the said dried solid composition is commonly named a lyophilisate. It has been checked that this post-titration drying step does not alter the advantageous characteristics of the product of the invention, i.e. physiological pH compatibility, even after re-dispersing the said dried solid composition or lyophilisate in water or a mineral buffer or an organic buffer.

In a Further embodiment, the present invention further includes various uses of a liquid or solid lipid dispersion according to the present invention, such as a solid composition (e.g. a composition of solid amorphous particles or a dried solid composition or lyophilisate) or an emulsion, including uses such as:
an anti-inflammatory agent or a component of an anti-inflammatory composition,
an anti-microbial agent or a component of an anti-microbial composition,
a cosmetic agent or a component of a cosmetic composition,
an emulsifier or a component of an emulsifying composition,
a detergent or a component of a detergent composition,
as a diagnostic reagent, and
as a vaccine adjuvant, especially for cytotoxic T lymphocyte induction, or a component of a vaccine composition; in the latter use, the dispersion of lipids of this invention may be admixed with an antigen and an immunopotentiatory amount of an immunogenicity inducing or enhancing compound; the vaccine composition may be administered orally, topically, epicutaneously, intramuscularly, intradermally, subcutaneously, intranasally, intravaginally, sublingually or via inhalation; for further details relating to such use, reference is made to “Vaccine adjuvants” (2000) ed. Derek T. O'Hagan, the content of which is incorporated herein by reference.

In all of the aforesaid uses, the lipid dispersion of this invention takes advantage of its pH compatibility characteristics. Additionally, the invention includes the use of a liquid or solid dispersion of lipids such as defined hereinabove for the manufacture of a medicament, e.g. as an ingredient of a pharmaceutical or veterinary composition.

In another embodiment, the present invention includes a synthetic vector or delivery vehicle characterised as being a combination of a dispersion of lipids such as previously disclosed and a biologically active molecule. Such synthetic vectors or delivery vehicles are useful in being able to introduce a wide range of biologically active molecules into a wide range of eukaryotic cells, preferably cells performing endocytosis or phagocytosis. In a first aspect of this embodiment, the biologically active molecule may be a therapeutic agent (such as defined hereinafter) which the lipids are able to transport over the membrane for introducing the said agent into the cell. In a second aspect of this embodiment, the biologically active molecule may be a macromolecule selected for instance from the group consisting of genetic material, polypeptides, glycosylated polypeptides, proteins, glycosylated proteins, protamine salts and sugars.

According to a particular embodiment the biologically active molecule of the invention is a polynucleotide. A polynucleotide may be a single stranded DNA or RNA, or a double-stranded DNA, RNA or DNA-RNA hybrid, but also refers to triple- or quadruple-stranded polynucleotides with therapeutic value. Examples of double-stranded polynucleotides include structural genes, preferably including operator control and termination regions, and self-replicating systems such as plasmid DNA as well as double stranded RNA which can be used for posttranscriptional gene silencing. Examples of single stranded polynucleotides which can be of therapeutic value include sense and antisense polynucleotides, ribozymes, triplex-forming oligonucleotides, and peptide nucleic acids. Preferably, such ‘therapeutic strands’ have one or more of its nucleotide linkages stabilized by non-phosphodiester linkages such as phosphorothiate or phosphoroselenates.

According to a particular embodiment of the invention, the bioactive molecule is a polynucleotide and the complex between the lipids of the present invention and the polynucleotide is stablized by addition of block copolymer surfactants. Examples of block copolymers are known in the art and include PEG-containing block polymers such as Pluronic® and poly-L-aspartamide.

Importantly the present invention provides for efficient introduction of genetic material into a wide range of cell types, preferably cells performing endocytosis or phagocytosis, for instance fibroblast cells. Within such vectors and delivery vehicles, the weight ratio of the lipids to the said biologically active molecule is preferably from about 1:15 to 15:1, more preferably from 1:2 to 5:1. As is well known in the art, practical considerations such as toxicity at high concentrations, potentially adverse interactions with the biological milieu, side effects, ability to reach tissues and the like will dictate the selection of an appropriate weight ratio in each case, depending on the specific macromolecule concerned.

In a further embodiment, the present invention includes a method for introducing a biologically active molecule into a eukaryotic cell, comprising bringing said molecule in contact with the dispersion of lipids as previously defined, in the presence of a culture medium containing the said eukaryotic cell. The type of cells concerned and the kind of macromolecules, especially genetic material, concerned in this embodiment are as disclosed with respect to the synthetic vectors hereinabove. When the said macromolecule is DNA, the biological material delivery method of the invention is preferably performed in the presence of a membrane permeability enhancing agent such as calcium phosphate. In another aspect of this embodiment, the present invention provides eukaryotic cells treated by the said method, i.e. transformed or transfected by means of a synthetic vector such as disclosed in detail hereinabove.

In another embodiment, the present invention further includes a pharmaceutical composition comprising an effective amount of a dispersion of lipids (such as previously defined), optionally in combination with a biologically active molecule, and optionally one or more pharmaceutically acceptable carriers. Such pharmaceutical compositions are useful for administration in a therapeutically effective amount to a mammal, for instance a human, in need of the biologically active macromolecule included in the said composition. The invention also provides a method of treatment of a mammal in need of a biologically active molecule, comprising administering to the said mammal a therapeutically effective amount of the above pharmaceutical composition. According to standard practice in the art, administration to the patient may be effected by any conventional means, i.e. for instance orally, intranasally, subcutaneously, intramuscularly, intradermally, intravenously, intraarterially, parenterally or by catheterization.

As used in the previous embodiments of the present invention, the term “biologically active molecule” includes both therapeutic agents and cosmetic agents for topical or subcutaneous administration. Within the said meaning, the therapeutic agent may be selected from the group consisting of anti-fungal agents, hormones, vitamins, peptides, enzymes, polypeptides, glycosylated polypeptides, proteins, glycosylated proteins, anti-allergic agents, anti-coagulation agents, anti-tubercular agents, antiviral agents, antibiotics, anti-bacterial agents, anti-inflammatory agents, anti-protozoan agents, local anesthetics, growth factors, cardiovascular agents, diuretics and radioactive compounds. In particular, the therapeutic agent may be selected from the group consisting of scopolamine, nicotine, methylnicotinate, mechlorisone dibutyrate, naloxone, caffeine, salicylic acid, and 4-cyanophenol.

Suitable anti-fungal agents include ketoconazole, nystatin, griseofulvin, flucytosine, miconazole and amphotericin B. Suitable hormones include growth hormone, melanocyte stimulating hormone, estradiol, cortisol, luteinizing hormone, follicle stimulating hormone, somatotropin, somatomedins, adreno-corticotropic hormone, parathormone, vasopressin, thyroxine and testosterone. Suitable vitamins include retinoids, retinol palmitate, ascorbic acid and α-tocopherol. Suitable peptides and enzymes include bombesin, cholecystokinin, insulin, gastrin, endorphins, enkephalins, prolactin, oxytocin, gonadotropin, corticotropin, β-lipotropin, γ-lipotropin, calcitonin, glucagon, thyrotropin, elastin, cyclosporin, manganese super oxide dismutase and alkaline phosphatase. Suitable anti-coagulation agents include heparin. Suitable anti-tubercular agents include paraminosalicylic acid, isoniazid, capreomycin sulfate cycloserine, ethambutol hydrochloride ethionamnide, pyrazinamide, rifampin, and streptomycin sulfate. Suitable antiviral agents include acyclovir, amantadine, azidothymidine, ribavirin and vidarabine monohydrate. Suitable antibiotics include dapsone, chloramphenicol, neomycin, cefaclor, cefadroxil, cephalexin, cephradine erythromycin, clindamycin, lincomycin, amoxicillin, ampicillin, bacampicillin, carbenicillin, dicloxacillin, cyclacillin, picloxacillin, hetacillin, methicillin, nafcillin, oxacillin, penicillin, ticarcillin, rifampin and tetracycline. Suitable anti-inflammatory agents include diflunisal, ibuprofen, indomethacin, meclofenamate, mefenamic acid, naproxen, oxyphenbutazone, phenylbutazone, piroxicam, sulindac, tolmetin, aspirin and salicylates. Suitable anti-protozoan agents include chloroquine, hydroxychloroquine, metronidazole, quinine and meglumine antimonate. Suitable local anesthetics include bupivacaine, chloroprocaine, etidocaine, lidocaine, mepivacaine, procaine and tetracaine and salts (such as hydrochloride) thereof. Suitable growth factors include Epidermal Growth Factor, Fibroblast Growth Factor, Insulin-Like Growth Factors, Nerve Growth Factor, Platelet-Derived Growth Factor, Stem Cell Factor, Transforming Growth Factors of the α family or the β family. Suitable cardiovascular agents include clonidine, propranolol, lidocaine, nicardipine and nitroglycerin. Suitable diuretics include mannitol and urea. Suitable radioactive compounds may include for instance a radioactive element selected from the group consisting of strontium, iodine, rhenium and yttrium.

Cosmetics suitable as biologically active molecules in this invention may be selected for instance from the group consisting of Vitamin A, Vitamin C, Vitamin D, Vitamin E, Vitamin K, β-carotene, collagen, elastin, retinoic acid, aloe vera, ointment bases (such as lanolin, squalene and the like), hyaluronic acid, sunscreen agents and nucleosides. Suitable sunscreen agents include for instance isobutyl p-aminobenzoate, diallyl trioleate, monoglyceryl p-aminobenzoate, propyleneglycol p-aminobenzoate, benzyl salicylate, benzyl cinnamate and mixtures thereof. When the biologically active molecule is a cosmetic agent, the pharmaceutical composition of the invention may take the form of a cosmetic cream, ointment, lotion, skin softener, gel, blush, eye-liner, mascara, acne-medication, cold cream, cleansing cream, or oleaginous foam.

Pharmaceutically acceptable carriers suitable for use in the pharmaceutical compositions of this invention include:
bacteriostatic agents such as quaternary ammonium compounds (including alkyldimethylbenzylammonium chlorides, cetylpyridinium chloride, cetyltrimethylammonium bromide, β-phenoxyethyl-dimethyldodecylammonium bromide and the like), benzoic acid, benzyl alcohol, p-hydroxybenzoic acid butyl ester or methyl ester, chloro-butanol, chlorocresol, phenol, potassium or sodium benzoate, potassium sorbate and sorbic acid;
antioxidants such as ascorbic acid and ascorbyl palmitate;
moisture content control agents and humectants;
suspending and viscosity-increasing agents such as agar, alginic acid, aluminum monostearate, bentonite, carbomers, carboxymethylcellulose calcium or sodium, carrageenan, microcrystalline cellulose, dextrin, gelatin, guar gum, hydroxyethylcellulose, hydroxypropylcellulose, hydroxypropylmethylcellulose, magnesium aluminum silicate, methylcellulose, pectin, polyethylene oxide, polyvinyl alcohol, polyvinylpyrrolidone, propylene glycol alginate, silicon dioxide, zinc oxide, sodium alginate tragacanth and xanthan gum;
skin absorption enhancing agents such as pyrrolidones, fatty acids, sulfoxides, amines, terpenes, terpenoids, urea, glycols and alcohols;
bases such as glycerol, propylene glycol, isopropyl myristate and polyethylene glycol; and
oleaginous vehicles, coloring agents or foaming agents.

The various embodiments of the present invention exhibit numerous advantages over lipid dispersions of the prior art, including:
the method for preparing the lipid dispersions of the invention is easy and inexpensive to implement and achieves liquid dispersions which have and retain a pH compatible with physiological pH within a temperature range (between about 2° C. and 40° C.) useful for most medical applications and which can easily be transformed into solid compositions, either dried (e.g. lyophilisates) or amorphous, or into emulsions retaining the latter property when re-dispersed in any physiological medium;
the lipid dispersions of the invention able to efficiently transfect a wide range of types of cells, especially cells performing endocytosis or phagocytosis; and
the lipid dispersions of the invention are therapeutically useful by themselves, either in vivo or in vitro, or can be included as formulation agents into a wide range of pharmaceutical compositions, diagnostic kits or cosmetic preparations.

The present invention will now be further explained by reference to the following working examples and comparative examples, which should in no way be interpreted as limiting its scope.

### EXAMPLES

### Example 1 (Comparative)

### Preparation of Amino-Amidine Liposomes in Water

N-terbutyl-N′-tetradecyl-3-tetradecyl-aminopropionamidine (having a melting point of 34° C.) is dispersed in water (injection grade available from BAXTER, Cat. Nr. ADA 0304) and kept overnight at 4° C. It is then dispersed at room temperature using a TV45 Ultra-Turrax blender (available from Jahnke & Kunkel) until a concentration of 3 mg/ml is achieved. The resulting dispersion was then poured into a M110S microfluidizer (available from Microfluidics International Corp., Newton, Mass.) and then processed at 45° C. for four cycles of two minutes each, the interaction chamber outlet being packed in ice. The resulting dispersions was cooled and then passed through a 0.2 μm filter (in order to eliminate large particles) and then packed in sterile vials and stored at 4° C. Stability at 4° C. was satisfactorily checked by means of turbidity measurements for over five weeks.

### Example 2

### Preparation and Control of Amidinium Salt Dispersions in Water

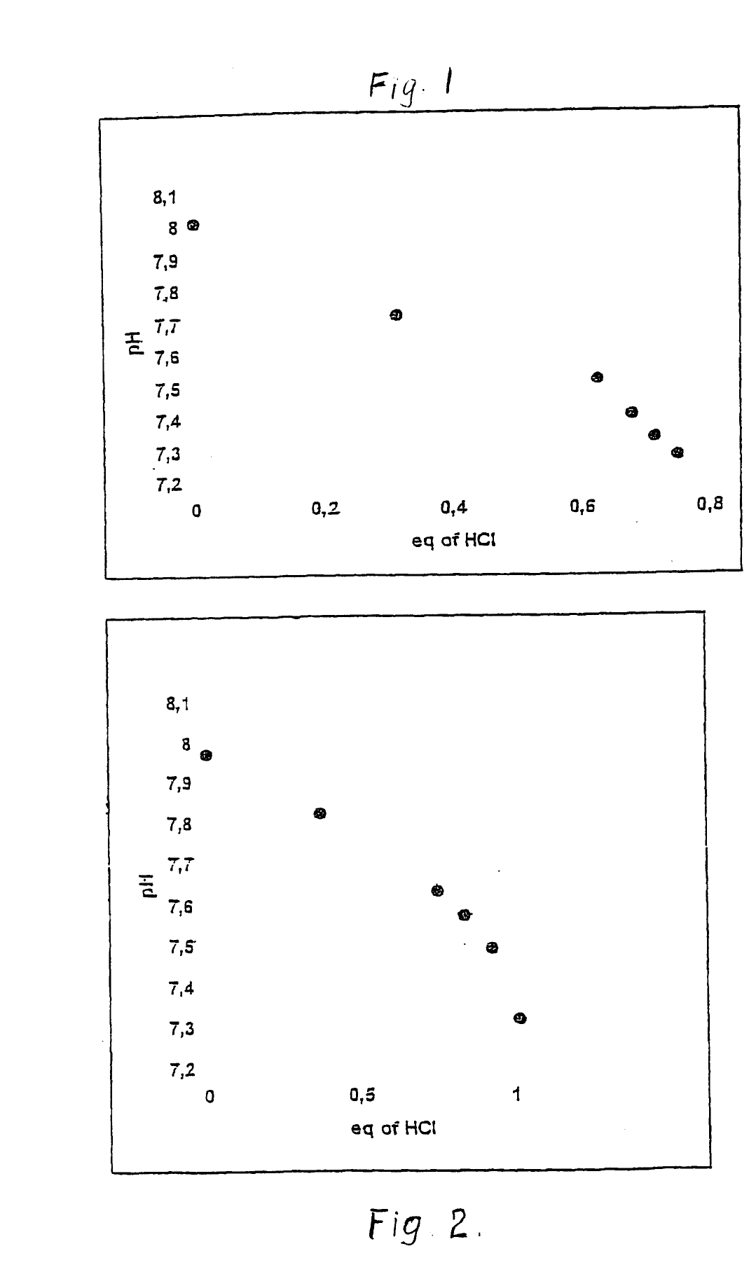

Immediately after the processing step disclosed in example 1, 1.1 mole equivalent of hydrochloric acid was added progressively to the microfluidized dispersion, pH of the solution being recorded at 25° C. as shown in FIG. 1.

After complete titration of the amidine function, the amidinium salt dispersion was processed again at 45° C. using the same M110S microfluidizer equipment as in example 1. At the end of this second processing step, the pH of the dispersion was measured as 7.3 at 25° C.

The resulting titrated liposomes were cooled and then passed through a 0.2 μm filter (in order to eliminate large particles) and an average liposome size of 90 nm was measured. Filtered liposomes were then packed in sterile vials and stored at 4° C. Their stability at 4° C. was checked by turbidity measurements for over five weeks, i.e. titration by a strong acid had no adverse effect on the stability of the dispersion. Cytotoxicity was determined by means of a (3-(4,5-dimethylthiazol-2-yl)-2,5-diphenyl tetrazolium bromide assay, using a kit commercially available from Promega Benelux (Leyden, The Netherlands). COS-7 monkey fibroblast cell survival was expressed as the amount of dye reduction relative to that of the untreated control cells. The titrated liposomes are not toxic on COS-7 cells, as determined by the Cytotox 96 non-radioactive cytotoxy assay G 1780.

### Example 3 (Comparative)

### Preparation of Amino-Amidine Dispersions in Sodium Phosphate Buffer

Preparation of the lipid dispersion was performed according to the procedure disclosed in example 1, except that the first step of N-terbutyl-N′-tetradecyl-3-tetradecylaminopropionamidine dispersion was effected in 20 mM sodium phosphate buffer (pH 7.3) instead of water.

### Example 4

### Preparation and Control of Amidinium Salt Liposomes in Sodium Phosphate Buffer

Preparation of the lipid dispersion was performed according to the procedure disclosed in example 2 except that, alike in example 3, the initial processing step of amino-amidine dispersion was effected in 20 mM sodium phosphate buffer (pH 7.3) instead of water. The pH of the solution was recorded at 25° C. as a function of the amount of hydrochloric acid added, as shown in FIG. 2. Long-term (i.e. more than five weeks) stability and cytotoxicity of the titrated liposomes were successfully checked according to the same methodology as disclosed in example 2.

### Example 5

### Transfection of Fibroblast Cells with Amino-Amidine Dispersions or Amidinium Salt Dispersions in the Presence of Protamine Sulfate

COS-7 monkey fibroblast cells were grown in a Dulbecco's Modified Eagle Medium (DMEM) culture medium supplemented with 10% heat-inactivated foetal bovine serum and antibiotic/antimytotic and maintained at 37° C. in a humidified 5% CO₂ incubator. The cells were then seeded, one day prior to transfection, in a 24-well culture dish and allowed to reach at least 50% confluency.

COS-7 monkey fibroblast cells were transfected with DNA/protamine sulfate/amidine complexes or DNA/protamine sulfate/amidinium salt complexes according to the following procedure. A plasmid DNA/protamine sulfate mixture (1:1 weight ratio) was mixed with either the amino-amidine liposomes of comparative examples 1 and 3 or the amidinium salt dispersions of examples 2 and 4, at a 1:2 DNA:lipids weight ratio in 20 mM sodium phosphate buffer at pH 7.3. After incubation at 23° C. for 15 minutes, 50 μl of the resulting DNA-lipid complex was mixed with 450 μl of DMEM and added to the cells for transfection. After two hours of incubation, the cell medium was changed with regular medium containing 10% heat inactivated foetal bovine serum (hereinafter referred as FBS). The cells were then incubated again for an additional 22 hours.

Functional transfection efficiency was then measured by means of a beta-galactosidase (hereinafter referred as β-GAL) activity assay as follows: cells were lysed by adding 250 μl of a lysis buffer (0.1 M potassium phosphate, 0.5% Triton® X-100, 0.1% deoxycholate, pH 7.0). The cell lysate (50 μl) was mixed with 50 μl o-nitrophenyl-β-D-galactopyranoside (1.54 mg/ml in 0.1 potassium phosphate buffer, pH 7.0) and incubated at 37° C. for 30 minutes. The reaction was terminated by adding 160 μl of 1 M Na₂CO₃ and absorbance was determined using a spectrophotometer at 405 nm. β-GAL activity was calculated using a β-GAL standard curve.

Table 1 indicates transfection efficiencies, expressed in IU (international units) per well, obtained for each of the dispersions prepared according to examples 1 to 4.

TABLE 1

| Example | 1 | 2 | 3 | 4 |
|---|---|---|---|---|
| IU/well | 0.0028 | 0.0589 | 0.0977 | 0.172 |

### Example 6 (Comparative)

### Preparation of Mixed Amino-Amidine/Dimyristoylphosphatidyl Choline Dispersions in Sodium Phosphate Buffer

Preparation of dispersions was performed according to the procedure disclosed in example 3, except that N-terbutyl-N′-tetradecyl-3-tetradecylamino-propionamidine was replaced by a mixture comprising 50% by weight of the said amino-amidine and 50% by weight dimyristoylphosphatidyl choline.

### Example 7

### Preparation of Mixed Amidinium Salt/Dimyristoylphosphatidyl Choline Dispersions in Sodium Phosphate Buffer

Preparation of dispersions was performed according to the procedure disclosed in example 4 except that, alike in example 6, N-terbutyl-N′-tetradecyl-3-tetradecylamino-propionamidine was replaced by a mixture comprising 50% by weight of the said amino-amidine and 50% by weight dimyristoylphosphatidyl choline. Long-term (i.e. more than five weeks) stability and cytotoxicity of the titrated dispersions were successfully checked according to the same methodology as disclosed in example 2.

### Example 8

### Transfection of Fibroblast Cells with Amino-Amidine Dispersions or Amidinium Salt Dispersions in the Absence of Protamine Sulfate

COS-7 monkey fibroblast cells were transfected according to the same experimental procedure as disclosed in example 5, except that: protamine sulfate was absent from the transfecting complexes.

Table 2 below indicates transfection efficiencies, measured as in example 5 and expressed in UI/well, obtained for each of the dispersions of examples 1-2 and 6-7.

TABLE 2

| Example | 1 | 2 | 6 | 7 |
|---|---|---|---|---|
| UI/well | 0.00267 | 0.058 | 0.0088 | 0.117 |

Results of examples 5 and 8 taken altogether clearly indicate that, whether in the presence or absence of protamine sulfate and whether in the presence or absence of a co-lipid in the dispersions, titration of the amidine function of the amino-amidine compound according to the procedure of the present invention makes it possible to multiply by a factor up to about 20 the transfection efficiency of DNA in fibroblast cells such as COS-7 monkey cells.

### Example 9

### Preparation of a Salt of the Amino-Amidine Compound and a Cosmotropic Anion

N-terbutyl-N′-tetradecyl-3-tetradecyl-aminopropionamidine (DiC14iPr) was dispersed at a concentration of about 9 mg/ml in cyclohexane containing 0.9% of methanol. This organic solution is lyophilised during 6 hours at a pressure of 0.1 hPa. The resulting powder is then dissolved in water at a concentration of 1 mg/mL, heated at 50° C. during 15 minutes and processed at 50° C. during 5 minutes using a T25 Ultra-thorax blender at 11000 rpm (available from VWR). It is then poured into a M110L microfluidizer (available from Microfluidics International Corp.). Aliquots of 0.1N phosphoric acid are added progressively to the solution during the processing in the microfluidizer at 50° C. until the pH value of the solution is 6.5-7.0 at 25° C. The amidinium salt solution is then lyophylised during 10 hours at a pressure of 0.1 hPa.

### Example 10

### Determination of the CMC of an Amidinium Salt

The amidinium salt of example 9 was dissolved at various concentrations in either water pH 7.0, PB (20 mM phosphate, pH 7.3) or in PBS (20 mM phosphate buffer, 150 mM NaCl, pH 7.3).

A langmuir balance (Krüss K12) was used to measure the surface tension of a solution of increasing amino-amidine concentration. A plot of the surface tension (mM/m) vs the logarithm of the concentration showed a sharp break toward a slope close to zero. The corresponding concentration was taken as CMC.

The CMC for the amino-amidinium compound associated with a cosmotropic phosphate anion was determined at 25° C. as 5×10⁻⁴ in water, 5.25×10⁻⁵M in PB and 6.75×10⁻⁵M in PBS.

### Example 11

### Effect of Protamine Sulfate on CMC

To determine the effect of protamine sulfate on the CMC protamine sulfate (PS) was added at a final concentration of 0.2 mg/mL to solutions of increasing concentration of the amidinium salt of Example 9.

The CMC for the amino-amidine compound associated with a cosmotropic anion in PBS+Protamine sulfate at 25° C. was determined as 2×10⁻⁵M.

### Example 12

### Transfection of Fibroblast Cells with Micellar Dispersions of an Amidinium Salt

Immediately before the transfection experiments, the amidinium salt of Example 9 was dissolved at a concentration of 0.1 mg/mL in water containing 5.0 g/l glucose and 0.1 mg/mL protamine sulfate. The solution was successively vortexed, heated during 15 minutes at 50° C. and then cooled at 20° C.

COS-7L cells (monkey fibroblasts) were grown in DMEM supplemented with 10% heat inactivated foetal bovine serum and penicellin 100 IU/mL-streptomycin 100 μg/mL, and were maintained at 37° C. in a humidified 5% incubator. One day prior to the transfection experiment, the cells were seeded in a 24-well culture dish and allowed to reach at least 80% confluency.

An amount of 10.4 μL of Plasmid DNA (pCMV-β, Clontech, 0.48 mg/mL) was mixed with 200 μl of the amino-amidine/protamine sulfate/glucose solution. After incubation at room temperature for 30 minutes, 42 μl of the DNA-lipids-protamine sulfate complex were mixed with 500 μl of DMEM without FBS and added to the cells for transfection. After a 3 hour incubation at 37° C. in a humidified 5% CO₂ incubator, the cell medium was changed with regular medium containing 10% heat inactivated FBS. The cells were then incubated for an additional 20 hours at 37° C. in a humidified 5% CO₂ incubator.

Transfection with Lipofectamine plus (available from Invitrogen, Merelbeke, Belgium) starting with the same amount of Plasmid DNA was performed as a comparative experiment, according to the manufacturer's instructions.

Transfection efficiency was measured in terms of beta-galactosidase activity using the beta-galactosidase activity assay described in Example 5 above. Cytotoxicity was determined as described in Example 2 above.

The results are provided in Table 3.

TABLE 3

| | β-gal relative | |
|---|---|---|
| Lipids | activity | Toxicity |
| Lipofectamine plus | 100% | 10-20% |
| DiC14iPr | 150% | None |

1. A dispersion of lipids in a dispersing medium, the said lipids comprising an amino-amidine compound A having the general formula:
R₁HN—(CH₂)ₙ—C(═NR₂)—NR₃R₄ (I)
wherein each of R₁, R₂, R₃ and R₄ is independently selected from the group consisting of hydrogen, C₃₋₂₀ alkyl, C₃₋₁₀ cycloalkyl, aryl and heteroaryl radicals, and n is an integer from 1 to 6 inclusive, wherein the amidine function of the said compound A is titrated substantially in water by means of an acid HX, wherein X is an anion, in a manner such that the pH of the said lipid dispersion is between about 6.5 and 7.8 within a temperature range from about 2° C. to 40° C.

2-36. (canceled)

37. The dispersion of lipids according to claim 1, wherein said anion X is a cosmotropic anion selected from the group consisting of sulfate, phosphate, acetate, citrate, and hydrogen-phosphate.

38. The dispersion of lipids according to claim 1, wherein said lipids are present in the form of liposomes or emulsion droplets or in the form of solid particles.

39. The dispersion of lipids according to claim 1, wherein said compound A is present in a concentration between 0.5×CMC and 100×CMC, CMC being the critical micellar concentration, and wherein said lipids are present in the form of micelles.

40. The dispersion of lipids according to claim 1, wherein the said dispersing medium comprises an organic buffer.

41. The dispersion of lipids according to claim 1, in a mixture with a polycationic peptide or polymer.

42. The dispersion of lipids according to claim 1, in a mixture with protamine sulfate.

43. The dispersion of lipids according to claim 1, wherein said compound A is selected from the group consisting of N-terbutyl-N′-tetradecyl-3-tetradecylaminopropionamidine, N-terbutyl-N′-dodecyl-3-dodecylamino-propionamidine, N-terbutyl-N′-hexadecyl-3-hexadecylaminopropion-amidine, N-terbutyl-N′-octadecyl-3-octadecylaminopropionamidine, N-isopropyl-N′-tetradecyl-3-tetradecylaminopropionamidine, N-isopropyl-N′-dodecyl-3-dodecylaminopropionamidine, N-isopropyl-N′-hexadecyl-3-hexa-decylaminopropionamidine and N-isopropyl-N′-octadecyl-3-octadecyl-aminopropionamidine.

44. The dispersion of lipids according to claim 1, in combination with a biologically active molecule and optionally with a pharmaceutically acceptable carrier.

45. The dispersion of lipids according to claim 1, in combination with a biologically active molecule and optionally with a pharmaceutically acceptable carrier, wherein said biologically active molecule is a macro-molecule selected from the group consisting of nucleic acids, DNA, RNA, mRNA, rRNA, tRNA, uRNA, ribozymes, antisense oligonucleotides, peptide nucleic acid (PNA), plasmid DNA, polypeptides, glycosylated polypeptides, proteins, glycosylated proteins, protamine salts and sugars.

46. The dispersion of lipids according to claim 1, in combination with a block copolymer surfactant.

47. A method of making a dispersion of lipids, comprising the steps of:
(a) dispersing an amino-amidine compound A having the general formula
R₁HN—(CH₂)ₙ—C(═NR₂)—NR₃R₄
wherein each of R₁, R₂, R₃ and R₄ is independently selected from the group consisting of hydrogen, C₃₋₂₀ alkyl, C₃₋₁₀ cycloalkyl, aryl and heteroaryl radicals, and n is an integer from 1 to 6 inclusive, in a liquid medium comprising an aqueous medium and optionally an organic solvent for compound A, and optionally an oily component,
(b) optionally processing the dispersion obtained in step (a) until vesicles comprising compound A is obtained, and
(c) titrating the dispersion obtained in step (a) or (b) with an acid HX, wherein X is an anion, and
(d) optionally processing the titrated dispersion obtained in step (c) until pH stabilisation
the said method being further characterised in that:
the aqueous medium of step (a) substantially consists of water, and
the acid HX is used in step (c) in an amount such as to substantially form an amidinium salt (A, HX) and such that the pH of the said lipid dispersion after titration is between about 7.0 and 7.6 within a temperature range from about 2° C. to 40° C.

48. The method according to claim 46, wherein said compound A is dispersed in step (a) in the presence of a lipid B, and wherein step (b) comprises processing said dispersion obtained in step (a) until vesicles comprising compound A and lipid B are obtained.

49. The method according to claim 46, further comprising the steps of:
(e) drying the titrated and optionally processed vesicles obtained in step (c) or (d) in order to obtain lipid solid particles,
(f) mixing the lipid solid particles obtained in step (e) with an oily component,
(g) rising the temperature of the mixture obtained in step (f) above the melting temperature of the lipid solid particles obtained in step (e) but not until the temperature of degradation of the said lipids, and
(h) emulsifying the mixture obtained in step (g) in the presence of water or a buffer.

50. The method according to claim 46, wherein said anion is a cosmotropic anion selected from the group consisting of sulfate, phosphate, acetate, citrate, and hydrogen-phosphate.

51. The method according to claim 46, wherein said compound A is selected from the group consisting of N-terbutyl-N′-tetradecyl-3-tetradecylaminopropionamidine, N-terbutyl-N′-dodecyl-3-dodecylamino-propionamidine, N-terbutyl-N′-hexadecyl-3-hexadecylaminopropion-amidine, N-terbutyl-N′-octadecyl-3-octadecylaminopropionamidine, N-isopropyl-N′-tetradecyl-3-tetradecylaminopropionamidine, N-isopropyl-N′-dodecyl-3-dodecylaminopropionamidine, N-isopropyl-N′-hexadecyl-3-hexa-decylaminopropionamidine and N-isopropyl-N′-octadecyl-3-octadecyl-aminopropionamidine.

52. A pharmaceutical composition comprising a dispersion of lipids in a dispersing medium, the said lipids comprising an amino-amidine compound A having the general formula:
R₁HN—(CH₂)ₙ—C(═NR₂)—NR₃R₄ (I)
wherein each of R₁, R₂, R₃ and R₄ is independently selected from the group consisting of hydrogen, C₃₋₂₀ alkyl, C₃₋₁₀ cycloalkyl, aryl and heteroaryl radicals, and n is an integer from 1 to 6 inclusive; and wherein said compound A is provided at a concentration around its critical micellar concentration (CMC), said CMC being between about 10⁻³ M and 10⁻⁶ M.

53. A pharmaceutical composition according to claim 52, wherein said compound A is selected from the group consisting of N-terbutyl-N′-tetradecyl-3-tetradecylaminopropionamidine, N-terbutyl-N′-dodecyl-3-dodecylaminopropionamidine, N-terbutyl-N′-hexadecyl-3-hexadecylamino-propionamidine, N-terbutyl-N′-octadecyl-3-octadecylaminopropionamidine, N-isopropyl-N′-tetradecyl-3-tetradecylaminopropionamidine, N-isopropyl-N′-dodecyl-3-dodecylaminopropionamidine, N-isopropyl-N′-hexadecyl-3-hexa-decylaminopropionamidine and N-isopropyl-N′-octadecyl-3-octadecylaminopropionamidine.
